(19) United States
(12) Patent Application Publication
(10) Pub. No.: US 2020/0016086 A1
(43) Pub. Date: Jan. 16, 2020

(54) Nanoparticulate Materials and Methods for Targeting Iron Acquisition and Metabolism for Treating Bacterial Infections

(71) Applicant: KENT STATE UNIVERSITY, Kent, OH (US)

(72) Inventors: Songping HUANG, Kent, OH (US); Min-Ho KIM, Hudson, OH (US); Zhongxia WANG, Nanchang (CN); Bing YU, Stow, OH (US); Huda ALAMRI, Stow, OH (US)

(21) Appl. No.: 16/485,661

(22) PCT Filed: Feb. 17, 2018
(86) PCT No.: PCT/US2018/018554

§ 371 Date: Aug. 13, 2019

Related U.S. Application Data
(60) Provisional application No. 62/460,432, filed on Feb. 17, 2017.

(51) Int. Cl. A61K 9/51; A61K 33/26; A01N 25/12; A01N 59/16

(52) U.S. Cl. CPC A61K 9/51; A61K 33/26; A61K 9/5192; A01N 59/16; A01N 25/12

(57) ABSTRACT

Novel biocompatible nanoparticles preferably based on a calcium or gallium analogue of Prussian blue, or independently an analogue of magnesium, or aluminum were designed and synthesized to take advantage of their ability to penetrate the bacterial cell membrane of the invading pathogen in an animal such as a human in both Gram-positive bacteria (e.g. Staphylococcus aureus) and Gram-negative bacteria (e.g. Pseudomonas aeruginosa), and undergo selective ion exchange with intracellular iron to disrupt iron metabolism in such pathogenic bacteria for antibacterial applications.

This invention was made with government support under Grant No. R01N015674 awarded by the National Institutes of Health—NINR. The Government has certain rights in the invention

### FIELD OF THE INVENTION

Novel biocompatible nanoparticles preferably based on a calcium or gallium analogue of Prussian blue, or independently an analogue of magnesium or aluminum were designed and synthesized to take advantage of their ability to penetrate the bacterial cell membrane in an animal such as a human in both Gram-positive bacteria (e.g. Staphylococcus aureus) and Gram-negative bacteria (e.g. Pseudomonas aeruginosa), and undergo selective ion exchange with intracellular iron to disrupt iron metabolism in such pathogenic bacteria for antibacterial applications.

### BACKGROUND OF THE INVENTION

Iron is an essential element for nearly all forms of life including pathogenic microorganisms. In the battle between the invading pathogenic microorganisms and the host vertebrates for this strategic micronutrient, evolution seems to have granted vertebrates the upper hand over pathogenic bacteria. For example, nature has developed sophisticated machinery in vertebrates to sequester, transport and store iron with an extremely high efficiency to tightly regulate the amount of free iron at both the systemic and cellular level, which significantly decreases the chance for the invading pathogenic bacteria to multiply and cause disease. In contrast, pathogenic microorganisms totally depend on the iron pool of the host vertebrate for iron supply and acquire iron by means of theft and robbery. At the time of infection, one of the host defense mechanisms is to withhold iron from invading pathogens. This iron withholding process, often referred to as nutritional immunity, mobilizes a score of iron transport and storage proteins to further restrict the availability of free iron. In response, pathogenic bacteria, after encountering iron starvation upon entering the host, will deploy a variety of equally sophisticated mechanisms with multiple redundant uptake routes that use siderophores and heme-uptake systems to evade nutritional immunity and gain access to host iron during infection, resulting in a constant arms race for iron between the host tissues and the invading pathogenic microorganisms. It is logical that external interventions that disrupt iron acquisition and metabolism in pathogenic bacterial cells will shift the balance of power in the battle and may constitute novel approaches to treating bacterial infections. However, use of synthetic iron chelators for intracellular iron depletion to exploit this vulnerability has thus far met with limited success as the naturally occurring siderophores form iron chelates with exceptional thermodynamic stability, which makes it difficult for the synthetic ligands to compete and displace iron from them. Furthermore, the typical synthetic iron chelators lack the ability to penetrate the bacterial cell membrane.

### SUMMARY OF THE INVENTION

In this invention, we disclose the antimicrobial properties of the novel nanoparticles (NPs) based on the calcium analogues of Prussian blue KCa[Fe^{III}(CN)₆].n H₂O and K₂Ca[Fe^{II}(CN)₆].nH₂O where n is from 1 to 24 (hereafter abbreviated as CaPB^{III} and CaPB^{II}). Such NPs can be readily internalized by the bacterial cell that invades animals and especially a human being and selectively deplete iron inside the bacterial cell by an ion-exchange reaction, thus inhibiting bacterial growth. In other words, depletion of intracellular iron is the mechanism of the antimicrobial action of such NPs. By the same token, nanoparticles having the formula KMg[Fe^{III}(CN)₆].nH₂O and K₂Mg[Fe^{II}(CN)₆].nH₂O where n is from 1 to 24 (hereafter abbreviated as MgPB^{III} and MgPB^{II}) also have the ability to disrupting iron acquisition and metabolism in bacteria by a similar ion-exchange reaction, and thus can be utilized for the applications described in the above. For the same purposes, the present invention further relates to nanoparticles having the formula Al[Fe^{III}(CN)₆].nH₂O and KAI[Fe^{II}(CN)₆].nH₂O where n is from 1 to 24 (hereafter abbreviated as AlPB^{III} and AlPB^{II}) that are capable of selectively deplete the intracellular iron in bacteria. The vulnerability of iron acquisition and metabolism in bacteria can also be exploited by delivering the so-called “fake” iron into bacterial cells using gallium compounds. The Ga³⁺ ion resembles the Fe³⁺ ion in terms of the ionic charge, ionic radius (r for Ga³⁺=0.62 Å vs. r for Fe³⁺=0.65 Å) and coordination number (i.e. CN=6), but is redox inactive, gallium is often dubbed as the “fake” iron. When gallium is given to bacteria as a micronutrient in place of iron, it will disturb the bacterial iron metabolism and thus imbibing their growth. Therefore, the gallium analogues of Prussian blue have the double-dipping mechanism of depleting iron on the one hand while delivering gallium into the bacterial cell to cause more severe disruption of iron acquisition and metabolism. Nanoparticles having the formula Ga[Fe^{III}(CN)₆].nH₂O and KGa[Fe^{II}(CN)₆].nH₂O where n is from 1 to 24 (hereafter abbreviated as GaPB^{III} and GaPB^{II}) can also be utilized. The same selective depleting intracellular iron reactions occur when using KCa[Fe^{III}(CN)₆].nH₂O, K₂Ca[Fe^{II}(CN)₆].nH₂O, KMg[Fe^{III}(CN)₆].nH₂O, K₂Mg[Fe^{II}(CN)₆].nH₂O, Al[Fe^{III}(CN)₆].nH₂O, and KAl[Fe^{II}(CN)₆].nH₂O.

The present invention further relates to a process for forming the above-noted nanoparticles comprising the reaction of:

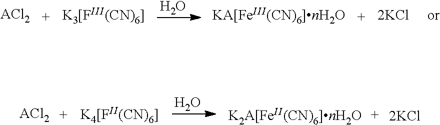

where A is Ca or Mg and n is from 1 to about 24. Similarly, the present invention further relates to a process for forming the above-noted nanoparticles comprising the reaction of:

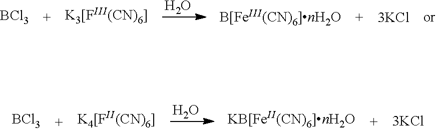

where B is Al or Ga and n is from 1 to about 24.

### BRIEF DESCRIPTION OF THE DRAWINGS

FIG. 1 shows a TEM image of the PVP-coated CaPB^{III} NPs (left) and a histogram of the size distribution (right);

FIG. 2 shows a single-crystal X-ray structure of KCa(H₂O)₂[Co^{III}(CN)₆], showing the 3D coordination polymer framework and the coordination environment of the Ca²⁺ ion (the K⁺ cation is omitted for clarity);

FIG. 3 shows a powder X-ray structure of K₂Ca[Fe^{II}(CN)₆], showing the 3D coordination polymer framework and the trapped K⁺ cations trapped inside the structural cavities;

FIG. 4 shows a powder X-ray structure of KGa[Fe^{II}(CN)₆], showing the 3D coordination polymer framework and the coordination of the Ga³⁺ and Fe²⁺ ions in two different octahedral centers;

FIG. 5 shows a selectivity of metal-ion removal (a) and kinetics of ion exchange between the Fe²⁺ ion and the CaPB^{III} NPs in aqueous solution;

FIG. 6 shows the effect of CaPB^{III} NPs on S. aureus cells: a) results of metal concentration analysis from the S. aureus cellular lysates after 24 hours of incubation; b) inhibiting curves of S. aureus cells incubated with various amounts of CaPB^{III} NPs (red), CaCl₂ (black) and K₃[Fe(CN)₆] (blue); c) results of time-dependent CaP^{III} NPs growth inhibition of S. aureus with different concentrations of CaPB^{III} NPs; and d) the OD₆₀₀ values of S. aureus cells incubated with CaPB^{III} NPs alone (black) and with CaPB^{III} NPs followed by addition of an equivalent amount of Fe to the cell culture after 2 hours incubation (red); and

FIG. 7 shows the results of cell viability studies in murine RAW 264.7 cells (black), human fibroblast cells (red), and endothelial cells (blue) incubated with different concentrations of CaPB^{III} NPs. Experiments were performed using MTT assay and incubated for 24 hours. *Mean value significantly different from that for the untreated CaPB NPs groups. P<0.05, n=3.

### DETAILED DESCRIPTION OF THE INVENTION

Synthesis of CaPB^{III} NPs:

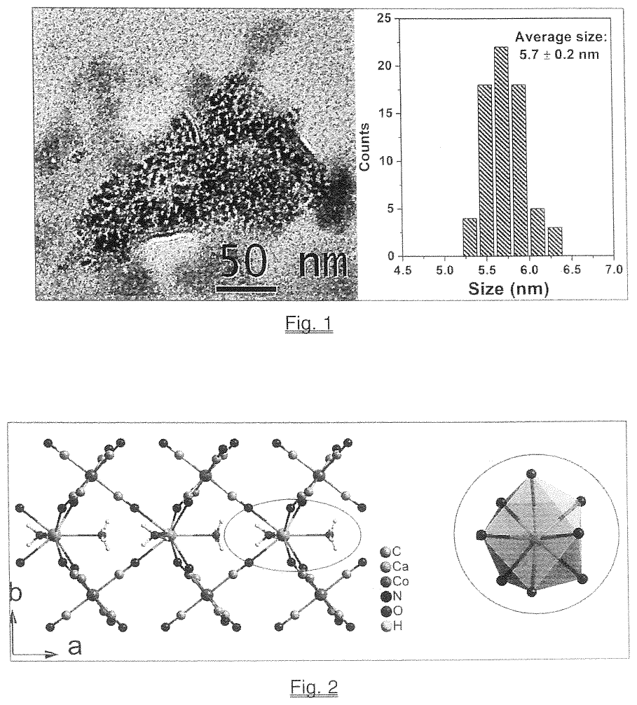

Aqueous solution 1.0 mM CaCl₂ (20 mL) containing 500 mg of polyvinylpyrrolidone (PVP with the average MW=40,000) was slowly added to a solution of 1.0 mM K₃[Fe(CN)₆] (20 mL) at room temperature, about 22° C., and atmospheric pressure, a clear pale-yellow solution was formed. After stirring for 20 minutes, the Tyndall effect detected by the use of a laser pointer indicated the formation of nanoparticles in the solution. In order to purify the nanoparticles, the solution was transferred into a dialysis bag made of regenerated cellulose tubular membrane (MWCO=3000) and dialyzed against distilled water for 4 hours. The solid product was collected by lyophilization. The metal analysis of this product using the atomic absorption spectrometric (AAS) method showed that the molar ratio of K:Ca:Fe is close to unity, confirming that the composition of the nanoparticle core has the expected formula KCa[Fe^{III}(CN)₆] and contained water molecules. The transmission electron microscopy (TEM) imaging studies revealed that the PVP-coated KCa[Fe^{III}(CN)₆] nanoparticles have near spherical shape with an average diameter of 5.7±0.2 nm as determined by counting and averaging the size of 64 NPs in the TEM picture frame (FIG. 1).

Furthermore, the energy dispersive X-ray spectroscopic (EDX) measurements showed the characteristic signals of Ca, Fe and K from several individual NPs randomly selected from the TEM grid (FIG. 1). Similarly, if MgCl₂ was utilized in the reaction, nanoparticles of KMg[Fe^{III}(CN)₆].nH₂O are formed. Since the reaction is invariably carried out in water, both the calcium and magnesium analogues will have bound water molecules in the formed framework structure. The reaction formula as set forth in the Summary of the Invention is as follows:

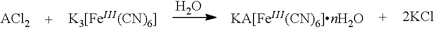

where A, independently, is Ca or Mg. Furthermore, K₂Ca[Fe^{II}(CN)₆].nH₂O or K₂Mg[Fe^{II}(CN)₆].nH₂O can be generated (when K₄[Fe^{II}(CN)₆] is used in the place of K₃[Fe^{III}(CN)₆]) where n is from 1 to 24. In a similar manner, in lieu of Ca or Mg cations, Al and Ga cations can be utilized in generally the same reaction format, i.e.

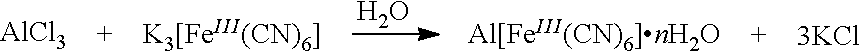

where n is from 1 to 24, or to yield Ga[Fe^{III}(CN)₆].nH₂O where n is from 1 to 24, or KAl[Fe^{II}(CN)₆].nH₂O (when K₄[Fe^{II}(CN)₆] is used in the place of K₃[Fe^{III}(CN)₆] where n is from 1 to 24, or KGa[Fe^{II}(CN)₆].nH₂O (when K₄[Fe^{II}(CN)₆] is used in the place of K₃[Fe^{III}(CN)₆]) where n is from 1 to 24, or any combination thereof.

The process for forming the nanoparticles containing calcium, magnesium, aluminum or gallium and ferricyanide (i.e. [Fe^{III}(CN)₆]³⁻) or ferrocyanide (i.e. [Fe^{II}(CN)₆]⁴⁻) ions comprises a reaction at a temperature of from about 0° C. to about 100° C., desirably from about 10° C. to about 80° C., and preferably from about 15° C. to about 60° C. The pressure is ambient atmospheric pressure. The amount of “n” per each molecule is from 1 to about 24, from 2 to about 10, and from about 2 to 4 or preferably about 3. The formed nanoparticles have a size of from about 2 to about 500 nanometers, desirably from about 2 to about 300 nanometers, and preferably from about 5 to about 150 nanometers.

The Fourier transform infrared (FTIR) spectra of the PVP-coated CaPB NPs exhibit a characteristic CN stretching vibration at 2094 cm⁻¹, a peak also observed in the bulk sample, in addition to the other characteristic stretching and bending vibrations attributable to PVP. To investigate the structure of this novel coordination polymer, we made numerous attempts to grow single crystals using the slow evaporation method under various conditions. To decrease the nucleation rate and prevent the formation of a precipitate, we attempted to crystallize the product in the acid conditions with pH=1, as this approach has been successfully used to crystallize many Prussian blue analogue compounds including the prototypical Prussian blue itself.

When the [Co^{III}(CN)₆]³⁻ anion was used as the surrogate for [Fe^{III}(CN)₆]³⁻, we obtained pale-yellow single crystals (H₃O)Ca(H₂O)₂[Co^{III}(CN)₆] (hereafter abbreviated as CaCoPB). The X-ray single crystal analysis showed that CaCoPB crystallizes in the orthorhombic crystal system (space group Pnma) with a=12.940(3) Å, b=13.697(3) Å, c=7.3605(2) Å and V=1304.6(5) Å³. The structure can be best viewed as consisting of Co³⁺ octahedra and Ca²⁺ biface-capped by six C atoms from trigonal prisms linked to form a 3D framework by the CN— groups. The Co atom is coordinated the CN— groups while the Ca atom is coordinated by six N atoms and furthermore capped by two 0 atoms of water molecules (see FIG. 2). Previously, we reported on the synthesis and MRI contrast properties of KGd(H₂O)₂[Fe^{II}(CN)₆].H₂O, an isostructural compound with the current CaCoPB phase. In KGd(H₂O)₂[Fe^{II}(CN)₆].H₂O, The K⁺ ion and a water molecule are located in the framework cavity, showing site-occupancy disorder on the same crystallographic position. In (H₃O)Ca(H₂O)₂[Co^{III}(CN)₆], this zeolitic water cavity is occupied by the hydronium H₃O⁺ ion. It is not unreasonable to assume that CaPB forms the same type of coordination polymer with a 3D network structure in which the Ca²⁺ ion is found in a similar biface-capped trigonal prism environment. The latter notion was readily confirmed by the TGA measurements of the bulk CaPB sample, which clearly showed the presence of two coordination water molecules and a zeolitic water molecule from the structural cavity, and hence indicating KCa(H₂O)₂[Fe^{III}(CN)₆].nH₂O as a formula for the NP core.

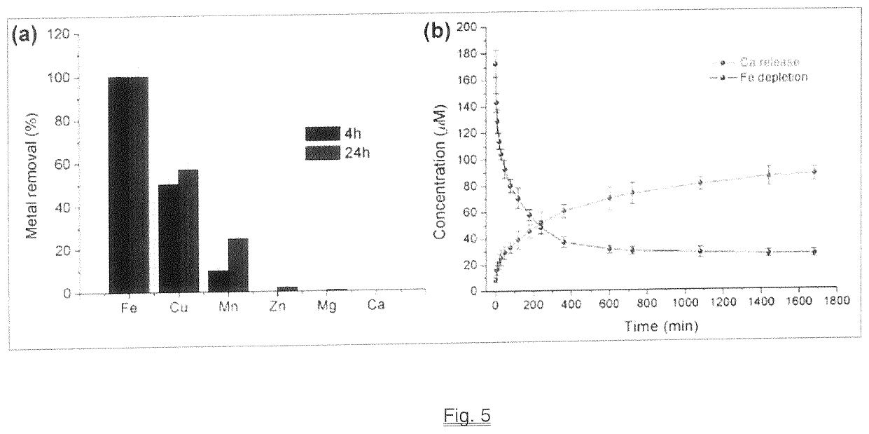

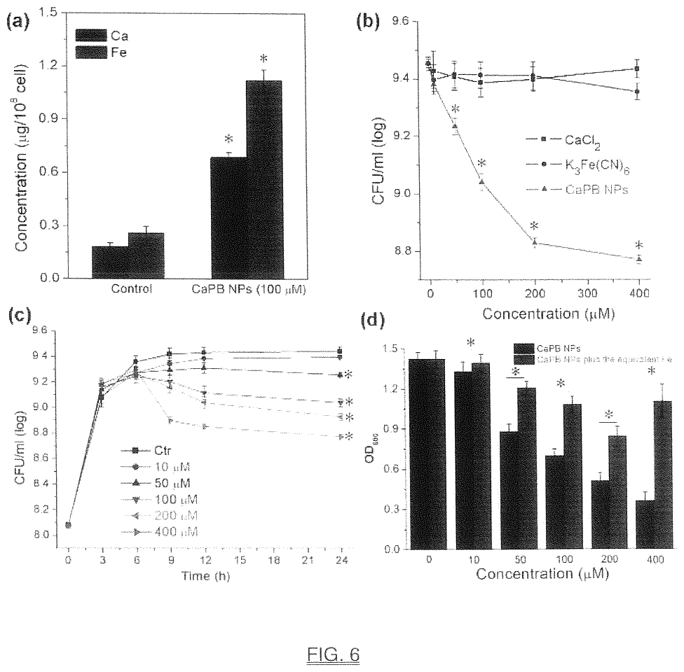

We studied the selectivity of Fe2+ depletion by CaPB NPs in the presence of other biologically relevant divalent metal ions including Cu²⁺, Mn²⁺, Zn²⁺, Mg²⁺ and Ca²⁺ each at the concentration level of 100 ppm in the same aqueous solution to compete with Fe²⁺ for ion exchange with the NPs over a time period of 24 h. The resulting selectivity for each ion was normalized against the removal of Fe²⁺ ions being set as 100%. The results were expressed as the percent removal for each ion. As depicted in FIG. 5a, CaPB NPs appeared to be the most selective toward the Fe²⁺ ion in the present of Cu²⁺, Mn²⁺ and Zn²⁺ ions.

The kinetics of Fe²⁺ removal was followed by measuring the decrease of Fe²⁺ ion concentration in aqueous solution where the dialysis bag containing the PVP-coated CaPB NPs was immersed. The simultaneous increase of Ca²⁺ ion concentration in the same solution was also determined quantitatively by AAS to verify the kinetics (FIG. 5b). The kinetics data can be fitted to two separate rate laws, i.e., a pseudo-first-order reaction up to the time point of 100 min with a rate constant of k₁=2.1×10⁻⁴ s⁻¹ and a half-life of T_{1/2}=55 min, and a second-order reaction with a rate constant of k₂=9.2×10⁻² M⁻¹ s⁻¹. Interestingly, the ion exchange can be visually followed by the color of the solution in the dialysis bag that turned pale yellow to blue in accordance with the time scale determined by the kinetic measurements, indicating an ion-exchange reaction between the CaPB NPs and Fe²⁺ ions to give Prussian blue.

X-Ray Single Crystal Structure Analysis:

KCa[Fe^{II}(CN)₆].nH₂O where n is from 1 to 24 is isostructural with KCa[Co^{III}(CN)₆].nH₂O where n is from 1 to 24 (hereafter abbreviated as CaCoPB). The X-ray single crystal analysis showed that CaCoPB crystallizes in the orthorhombic crystal system (space group Pnma) with a=12.940(3) Å, b=13.697(3) Å, c=7.3605(2) Å and V=1304.6(5) Å³. The structure can be best viewed as consisting of Co³⁺ octahedra formed from six C atoms of the CN⁻ groups and Ca²⁺ biface-capped prisms formed from six N atoms of the CN⁻ groups and two water molecules to form a 3D framework (see FIG. 2).

X-Ray Powder Structure Analysis:

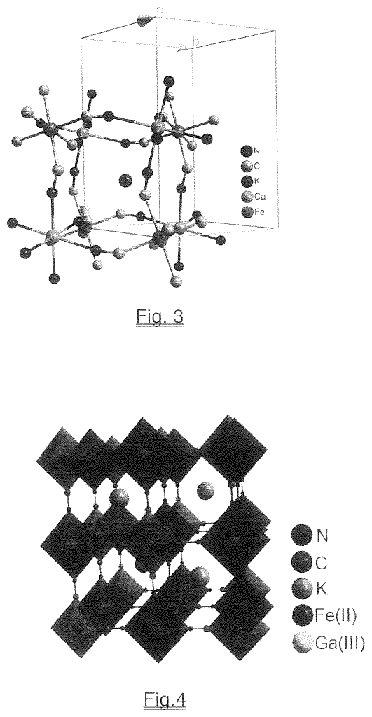

The crystal structure of K₂Ca[Fe^{II}(CN)₆].nH₂O where n is from 1 to 24 was determined by the X-ray powder diffraction technique. Compound K₂Ca[Fe^{II}(CN)₆].nH₂O crystallizes in the monoclinic crystal system (space group P2₁/n) with a=10.2941 (4) Å, b=7.50950 (31) Å, c=7.06254 (27) Å, β=90.3285 (20)° and V=545.95 (5) Å³. The structure is also consisting of Fe³⁺ octahedra formed from six C atoms of the CN⁻ groups and Ca²⁺ octahedra formed from six N atoms of the CN⁻ groups to form a 3D framework. There are large cavities created by the two different types of octahedra that share faces with each other. The K⁺ ions are located inside such cavities (see FIG. 3).

X-Ray Powder Structure Analysis:

The crystal structure of KGa[Fe^{II}(CN)₆].nH₂O where n is from 1 to 24 was determined by the X-ray powder diffraction technique. Compound KGa[Fe^{II}(CN)₆].nH₂O crystallizes in the cubic crystal system (space group Fm3̅m) with a=10.1856(1) Å and V=1056.72(5) Å³. The structure can be best described as the face-centered cubic structure defined by Fe²⁺ ions with Ga³⁺ ions occupying the octahedral holes. The infinite 3D framework structure is then completed by the coordination of the CN⁻ groups with the C atoms bound to Fe²⁺ ions and N atoms bound to Ga³⁺ ions. In addition, half of the tetrahedral sites in the crystal structure are occupied by the K ions (see FIG. 4).

X-Ray Powder Structure Analysis:

The crystal structure of Ga[Fe^{III}(CN)₆].nH₂O where n is from 1 to 24 was determined by the X-ray powder diffraction technique. Compound Ga[Fe^{III}(CN)₆].nH₂O is isostructural with KGa[Fe^{II}(CN)₆].nH₂O except that no K ions are found in the structure. The structure can also be described as the face-centered cubic structure defined by Fe³⁺ ions with Ga³⁺ ions occupying the octahedral holes. The infinite 3D framework structure is then completed by the coordination of the CN⁻ groups with the C atoms bound to Fe³⁺ ions and N atoms bound to Ga³⁺ ions. There are no K⁺ ions found in the structure.

Ion-Exchange of CaPB^{III} NPs with Fe²⁺ in Aqueous Solution:

the selectivity of Fe²⁺ depletion by CaPB^{III} NPs in the presence of other biologically relevant divalent metal ions including Cu²⁺, Mn^{2+,} Zn^{2+,} Mg²⁺ and Ca²⁺ in the same aqueous solution to compete with Fe²⁺ for ion exchange with the NPs over a time period of 24 hour was determined. The resulting selectivity for each ion was normalized against the removal of Fe²⁺ ions being set as 100%. The results were expressed as the percent removal for each ion. As depicted in FIG. 6a, CaPB^{III} NPs appeared to be the most selective toward the Fe²⁺ ion in the present of Cu²⁺, Mn²⁺ and Zn²⁺ ions. The observed selectivity is more or less consistent with that predicted by the Irving-Williams series, i.e. Mn(II)<Co(II)<Zn(II)>Cu(II), except for Fe(II) that forms the exceptionally stable Prussian blue from this ion-exchange reaction (FIG. 5a).

The kinetics of Fe²⁺ removal was measured and showed that there is a simultaneous release of Ca²⁺ ions from the CaPB^{III} NPs to the solution (FIG. 5b). The kinetics data can be fitted to two separate rate laws, i.e., a pseudo-first-order reaction up to the time point of 100 min with a rate constant of k₁=2.1×10⁻⁴ s⁻¹ and a half-life of T_{1/2}=55 min, and a second-order reaction with a rate constant of k₂=9.2×10⁻² M⁻¹ s⁻¹.

Antibacterial Properties of CaPB^{III} NPs:

to investigate cellular uptake, intracellular iron depletion, and antimicrobial properties of the PVP-coated CaPB^{III} NPs, we used the opportunistic pathogen Staphylococcus aureus (S. aureus) as an in vitro bacterial cellular model. S. aureus is a Gram-positive coccoid bacterium that causes a spectrum of ailments ranging from wound infections to more severe diseases such as respiratory infections and food poisoning. For cellular uptake studies, we incubated the bacteria in 14-mL bacterial culture tubes with a TSB medium containing 100-μM CaPB NPs and 20-μL PBS for 24 hours in parallel with the bacteria cultured with TSB supplemented with 20-μL PBS as the control. Both the optical density (O.D.) values prior to cellular lysis and metal concentrations in cellular lysates were determined, and hence the calcium and iron concentrations were expressed as concentration per cell in femtogram.

Nonetheless, the increase of both the calcium and iron concentration in the cellular lysates provided sufficiently strong evidence to confirm the internalization of CaPB NPs in S.A. cells. Lethal dosage with respect to various types of bacteria usually range from about 1 micromole to about 1,000 micromoles, and preferably from about 10 micromoles to about 500 micromoles per liter, also known as micromolar concentration uM.

In the above studies, colony-forming unit (CFU) values of bacterial cells treated with CaPB^{III} NPs were first determined, followed by cellular lysis performed by the use of concentrated nitric acid in order to quantitatively analyze the metal concentrations. Both calcium and iron in cellular lysates were then measured by AAS to monitor the cellular uptake of CaPB^{III} NPs. The calcium and iron concentrations were expressed as concentration per 10⁸ cells in microgram. As shown in FIG. 6a, there was a significant increase of both the calcium (i.e. ca. 5 times higher than the control) and iron (i.e. ca. 4 times higher than the control) concentration. Nonetheless, the increase of both calcium and iron in cellular lysates provided sufficiently strong evidence to confirm the internalization of CaPB^{III} NPs in S. aureus cells. The amount of iron removed from bacteria cells range from 0.01 μg/10⁸ cells to 100 μg/10⁸.]

For bacterial cytotoxicity studies, we incubated bacteria with the TSB medium containing various amounts of CaPB^{III} NPs in comparison with the bacteria cultured with the corresponding amounts of either Ca²⁺ ions or [Fe^{II}(CN)₆]³⁻ ions, the two constituent components in the CaPB^{III} NPs, as two separate controls. As shown in FIG. 6b, CaPB^{III} NPs inhibited the growth of S. aureus in a dose-dependent manner at concentrations greater than 10 μM, while either CaCl₂) or K₃[Fe(CN)₆] almost has no effect on growth at the concentration range of 10 μM to 400□μM. In addition, we also tested the time-dependent growth inhibition curve for CaPB^{III} NPs (FIG. 6c). There is a pronounced effect on the bacterial growth after 9 hours of incubation (FIG. 6c). As in FIG. 6b, CaPB NPs inhibited the growth of S.A. in a dose-dependent manner at concentrations greater than 10 μM with an estimated LD50 of ca. 50 μM, while both CaCl₂ and K₃[Fe(CN)₆] have no effect on growth.

To ascertain that such a striking antibacterial action of the CaPB NPs was attributable to the cellular iron depletion or sequestration by the internalized NPs, we carried out a rescue test by replenishing the cell cultures with an amount of iron corresponding to each concentration of CaPB Ns given to the cells for two hours. As shown in FIG. 6d, iron addition to the cell cultures reversed the growth-inhibitory effect by these NPs. However, for the cells treated with higher concentrations of NPs (i.e. >50 μM), full restoration of growth became unachievable, suggesting some irreversible damage might have been done to the iron metabolism of the cell after they were treated with higher concentrations of NPs for 24 hours.

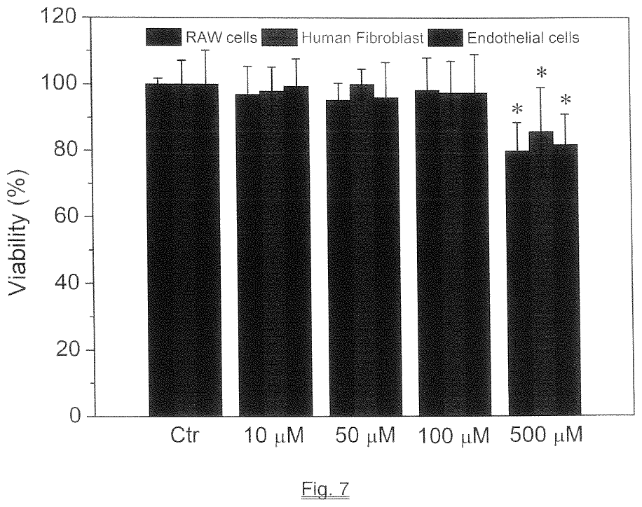

To evaluate the cytotoxicity of CaPB^{III} NPs in mammalian cells, we incubated murine RAW 264.7 macrophage-like cells, normal human fibroblast cells and human umbilical endothelial cells with various concentrations of NPs in the range of 10 μM to 500 μM. The cell viability was all assayed using the MTT method. As seen in FIG. 7, after incubation for 24 hours with 500 μM PVP-coated CaPB^{III} NPs, i.e. approximately ten times of the LD₅₀ value in S. aureus, more than 80% of the cells remained viable for all the three mammalian cell lines, indicating that such NPs are less toxic in mammalian cells. More importantly, when examined under the microscope, RAW cells and human fibroblast cells treated with such NPs all appeared to be healthy and viable with no discernable morphological change, which indicates that a wide therapeutic window may exist for the use of such NPs to treat bacterial infections. In other words, the therapeutic index (TI) of CaPB^{III} NPs should be relatively large, i.e. TI>10.

In summary, our approach to using PVP-coated CaPB NPs to selectively deplete cellular iron in bacteria offers a unique opportunity to design and develop novel antimicrobial agents for treating bacterial infections, particularly those caused by the multidrug-resistant organisms (MDROs). The currently known mechanisms of bacterial resistance to antibiotics include: (1) the enzymatic degradation of antibacterial drug molecules, (2) an alteration of bacterial proteins targeted by antibacterial drugs to reduce their binding capacity, and (3) a change in membrane permeability to antibacterial drugs by either decreasing permeability or increasing active efflux of antibacterial drugs. However, the current iron-depleting strategy may not be susceptible to any of these classical mechanisms, and thus defeating drug resistance in the MDROs. Therefore, the ability to target iron acquisition and metabolism in pathogenic bacterial cells using NPs such as these demonstrated in this communication may represent a paradigm shift in treating bacterial infections.

While in accordance with the patent statutes, the best mode and preferred embodiment have been set forth, the scope of the invention is not limited thereto, but rather by the scope of the attached claims.

What is claimed is:

1. A biocompatible nanoparticle comprising:
a compound having the formula KA[Fe^{III}(CN)₆].nH₂O wherein A is Ca or Mg and n, independently, is from 1 to about 24, and wherein the particle size of said compound is from about 2 to about 500 nanometers; or a compound having the formula B[Fe^{III}(CN)₆].nH₂O wherein B is Al or Ga, and n, independently, is from 1 to about 24, and wherein the particle size of said compound is from about 2 to about 500 nanometers.

2. The biocompatible nanoparticle of claim 1, wherein n, independently, is from about 2 to about 10, and wherein said particle size, independently, is from about 2 to about 300 nanometers.

3. The biocompatible nanoparticle of claim 2, wherein n, independently, is from about 2 to about 4, and wherein said particle size, independently, is from about 3 to about 150 nanometers.

4. The biocompatible nanoparticle of claim 2, wherein said nanoparticle, independently, is capable of selectively depleting intracellular iron from a bacterial cell.

5. A biocompatible nanoparticle comprising:
a compound having the formula K₂A[Fe^{II}(CN)₆].nH₂O wherein A is Ca or Mg and n, independently, is from 1 to 24; and wherein said particle size, independently, is from about 2 to about 500 nanometers; or a compound having the formula KB[Fe^{II}(CN)₆].nH₂O wherein B is Al or Ga, and n, independently, is from 1 to about 24, and wherein said particle size, independently, is from about 2 to about 500 nanometers.

6. The biocompatible nanoparticle of claim 5, wherein n, independently, is from about 2 to about 10, and wherein said particle size, independently, is from about 2 to about 300 nanometers.

7. The biocompatible nanoparticle of claim 6, wherein n, independently, is from about 2 to about 4, and wherein said particle size, independently, is from about 3 to about 150 nanometers.

8. The biocompatible nanoparticle of claim 6, wherein said nanoparticle, independently, is capable of selectively depleting intracellular iron from a bacterial cell.

9. A process for preparing compatible nanoparticles having the formula KA[Fe^{III}(CN)₆].nH₂O, comprising the step of reacting
ACl₂ with 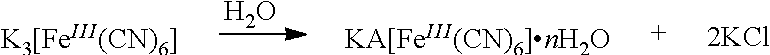
where A is Ca or Mg and n, independently, is from 1 to about 24.

10. The process of claim 9, wherein the reaction temperature is from about 0° C. to about 100° C., wherein n, independently, is from 2 to about 10, and wherein the average particle size of said KA[Fe^{III}(CN)₆].nH₂O, is from about 2 to about 500 nanometers; and wherein said nanoparticle is capable of selectively depleting intracellular iron in a human.

11. The process of claim 9, wherein the reaction temperature is from about 15° C. to about 60° C., wherein n, independently, is from 2 to about 4, and wherein the average particle size of said KA[Fe^{III}(CN)₆].nH₂O, is from about 3 to about 150 nanometers; wherein said nanoparticle is capable of selectively depleting intracellular iron in a human, and wherein said nanoparticle has a hydrophilic coating thereon.

12. A process for preparing compatible nanoparticles having the formula K₂A[Fe^{II}(CN)₆].nH₂O, comprising the step of reacting
ACl₂ with 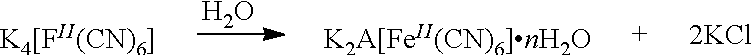
where A is Ca or Mg and n, independently, is from 1 to about 24.

13. The process of claim 12, wherein the reaction temperature is from about 0° C. to about 100° C., wherein n, independently, is from 2 to about 10, and wherein the average particle size of said K₂A[Fe^{II}(CN)₆].nH₂O, is from about 2 to about 500 nanometers; and wherein said nanoparticle is capable of selectively depleting intracellular iron in a human.

14. The process of claim 12, wherein the reaction temperature is from about 15′C to about 60° C., wherein n, independently, is from 2 to about 4, and wherein the average particle size of said K₂A[Fe^{II}(CN)₆].nH₂O, is from about 3 to about 150 nanometers; wherein said nanoparticle is capable of selectively depleting intracellular iron in a human, and wherein said nanoparticle has a hydrophilic coating thereon.

15. A process for preparing compatible nanoparticles having the formula B[Fe^{III}(CN)₆].nH₂O, comprising the step of reacting
BCl₃ with 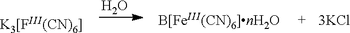
where B is Al or Ga and n, independently, is from 1 to about 24.

16. The process of claim 15, wherein the reaction temperature is from about 0° C. to about 100° C., wherein n, independently, is from 2 to about 10, and wherein the average particle size of said B[Fe^{III}(CN)₆].nH₂O, is from about 2 to about 500 nanometers; and wherein said nanoparticle is capable of selectively depleting intracellular iron in a human.

17. The process of claim 15, wherein the reaction temperature is from about 15° C. to about 60° C., wherein n, independently, is from 2 to about 4, and wherein the average particle size of said B[Fe^{III}(CN)₆].nH₂O, is from about 3 to about 150 nanometers; wherein said nanoparticle is capable of selectively depleting intracellular iron in a human, and wherein said nanoparticle has a hydrophilic coating thereon.

18. A process for preparing compatible nanoparticles having the formula KB[Fe^{II}(CN)₆].nH₂O, comprising the step of reacting
BCl₃ with 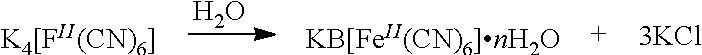
where A is Al or Ga and n, independently, is from 1 to about 24.

19. The process of claim 18, wherein the reaction temperature is from about 0° C. to about 100° C., wherein n, independently, is from 2 to about 10, and wherein the average particle size of said KB[Fe^{III}(CN)₆].nH₂O, is from about 2 to about 500 nanometers; and wherein said nanoparticle is capable of selectively depleting intracellular iron in a human.

20. The process of claim 18, wherein the reaction temperature is from about 15° C. to about 60° C., wherein n, independently, is from 2 to about 4, and wherein the average particle size of said KB[Fe^{II}(CN)₆].nH₂O, is from about 3 to about 150 nanometers; wherein said nanoparticle is capable of selectively depleting intracellular iron in a human, and wherein said nanoparticle has a hydrophilic coating thereon.
